(19) United States
(12) Patent Application Publication
(10) Pub. No.: US 2022/0233133 A1
(43) Pub. Date: Jul. 28, 2022

(54) HUMAN TACTILE PREPULSE INHIBITION ASSAY

(71) Applicant: President and Fellows of Harvard College, Cambridge, MA (US)

(72) Inventors: David D. Ginty, Cambridge, MA (US); Lauren L. Orefice, Cambridge, MA (US)

(73) Assignee: President and Fellows of Harvard College, Cambridge, MA (US)

(21) Appl. No.: 17/612,855

(22) PCT Filed: May 21, 2020
(86) PCT No.: PCT/US2020/033984

§ 371 Date: Nov. 19, 2021

Related U.S. Application Data
(60) Provisional application No. 62/851,374, filed on May 22, 2019.

(51) Int. Cl. A61B 5/00; A61B 5/395; A61B 3/11; A61B 5/0533; G16H 10/20

(52) U.S. Cl. CPC A61B 5/4035; A61B 5/395; A61B 3/112; A61B 2503/06; A61B 5/4842; A61B 5/4848; G16H 10/20; A61B 5/0533

(57) ABSTRACT

In some aspects, the disclosure relates to a prepulse inhibition (PPI) assay comprising the steps of, administering a tactile prepulse to a human subject, administering a startle stimulus to the subject, and measuring the subject's response to the startle stimulus. In another aspect, the disclosure relates to a method for evaluating tactile hypersensitivity and/or sensorimotor impairment in a human subject, comprising, administering to the subject a PPI assay according to any one of the preceding claims, comparing the assay results to neuro-typical controls, and determining the degree of tactile hypersensitivity and/or sensorimotor impairment in the subject.

### GOVERNMENT SUPPORT

This invention was made with government support under NS097344 awarded by the National Institutes of Health. The government has certain rights in the invention.

### BACKGROUND

Autism Spectrum Disorder (ASD) is a range of mental disorders of neurodevelopmental origin. The cause of ASD is uncertain and is correlated with a variety of risk factors. ASD is divided in to two types, syndromic and non-syndromic, with syndromic often characterized by a pattern of somatic abnormalities and a neurobehavioral phenotype. The Centers for Disease Control and Prevention currently estimates the prevalence of ASD in the United States of America at 1 in 68 children, a number greater than are affected by diabetes, AIDS, cancer, cerebral palsy, cystic fibrosis, muscular dystrophy or Down syndrome—combined. A 2008 snapshot of 14 monitoring sites found a 78% increase in autism over the previous five years, and a 10-fold increase in reported prevalence over the last four decades. In the United States of America alone, estimated ASD-related costs exceed $230 billion per year and about $3.2 million to care for a person with autism over their lifetime. Given this increase in prevalence and the lack of evidence to suggest that patients with ASD have a shorter life expectancy than the general population, interventions that relieve ASD symptoms for both children and adults are urgently needed. There are currently no treatments approved by the U.S. Food and Drug Administration (FDA) for ASD. However, the development of novel therapeutic interventions will require early and reliable biomarkers and improved understanding of the underlying ASD pathophysiology that would allow for moving research towards therapeutic goals.

Most of the research on ASD so far has focused on mechanisms and circuits specific to the central nervous system, with little attention to the roles of the peripheral nervous system and spinal cord in ASD pathophysiology and core symptoms. There are, however, clues that point to an extra-cerebral locus of dysfunction in ASD. Critically, most ASD patients exhibit aberrant reactivity to sensory stimuli, including tactile stimuli. As originally observed by Leo Kanner in a landmark description of patients with autism, aberrant sensory reactivity is associated with ASD and has been recently added as an ASD diagnostic criterion (DSM-V). ASD patients often report altered tactile sensitivity in both glabrous (smooth) and hairy skin. More than 94% of children with ASD report hyper- and/or hypo-sensitivities in multiple sensory domains, and 61% of patients exhibit abnormalities in somatosensation. Moreover, the degree of somatosensory impairment is strongly correlated with increased anxiety behaviors and impairments in social behaviors. Specifically, the highest degree of impairment in neural processing of affective touch is associated with the greatest expression of ASD traits. However, the underlying neural mechanisms of tactile hypersensitivity in ASD are not known. Understanding how abnormalities in somatosensory processing influence brain development and behavior is crucial to understanding and treating ASD. Moreover, reducing tactile hypersensitivity is a large, unmet need for patients with ASD, which as predicted in recent rodent preclinical studies, may improve anxiety and other core symptoms of ASD. Because the methodologies for measuring tactile sensitivity vary widely across clinical and basic research fields, there is an urgent need for direct and objective sensory reactivity metrics in clinical studies to diagnose and assess deficits in ASD patients, and for designing effective therapeutic strategies.

### SUMMARY

Accordingly, one aspect of the present disclosure provides a tactile prepulse inhibition (TPPI) assay, which comprises the steps of: (a) administering a tactile prepulse to a human subject; (b) administering a startle stimulus to the subject; and (c) measuring the subject's response to the startle stimulus.

In certain embodiments, the inter-stimulus interval (ISI) between steps (a) and (b) is between about 15 milliseconds (ms) to about 1000 ms, inclusive. In certain embodiments, the ISI is about 250 ms.

In certain embodiments, the tactile prepulse is an air puff. The air puff is directed at the subject's skin. In certain embodiments, the air puff is directed at the subject's forearm. In certain embodiments, the air puff is directed at the hairy skin on the back of the subject's hand. In certain embodiments, the air puff is directed at the glabrous skin on the palm of the subject's hand. In certain embodiments, the air puff is directed at the subject's thigh. In certain embodiments, the air puff is directed at the subject's leg. In certain embodiments, the air puff is directed at the subject's foot. In certain embodiments, the air puff is directed at the subject's neck.

In certain embodiments, the air puff has a pressure which is greater than about 0.1 pounds per square inch (PSI) and less than or equal to 10 PSI. In certain embodiments, the air puff has a pressure of about 0.9 PSI. In a particular embodiment, the air puff has a pressure of about 2 PSI. In a particular embodiment, the air puff has a pressure of about 3 PSI.

In certain embodiments, the startle stimulus has an intensity of about 100 to about 150 decibels (dB). In a particular embodiment, the startle stimulus has an intensity of about 115 dB.

In certain embodiments, the subject's response to the startle stimulus is measured by electromyography (EMG). In certain embodiments, the subject's response to the startle stimulus is measured by eye-blink reflex. In certain embodiments, the subject's response to the startle stimulus is measured by the subject's autonomic response. In certain embodiments, the subject's response to the startle stimulus is measured by pupillometry. In certain embodiments, the subject's response to the startle stimulus is measured by galvanic skin resistance.

In certain embodiments, the subject is wearing a noise-canceling auditory device prior to and during the assay. The noise-canceling auditory device may be noise-canceling headphones.

Another aspect of the present disclosure provides a method for evaluating tactile hypersensitivity and/or sensorimotor impairment in a human subject, comprising: (a) administering to the subject a TPPI assay according to any one of the methods disclosed herein; (b) comparing the assay results to neuro-typical controls; and (c) determining the degree of tactile hypersensitivity and/or sensorimotor impairment in the subject. In certain embodiments, the method further comprises the step of (d) adjusting a treatment of tactile hypersensitivity and/or sensorimotor impairment in the subject.

In certain embodiments, the subject has been diagnosed with Autism Spectrum Disorder (ASD), Rett Syndrome (RTT), Phelan McDermid syndrome (PMS), Fragile X Syndrome, Neurofibromatosis, or Tuberous Sclerosis complex.

In certain embodiments, the treatment is a pharmacological treatment, such a small molecule drug, a large molecule drug (e.g., an oligosaccharide), or a biologic drug (e.g., a peptide, antibody, or oligonucleotide). In certain embodiments, the pharmacological treatment comprises the administration of a GABA_{A} agent (e.g., a GABA reuptake inhibitor or transport inhibitor, a GABA_{A} receptor agonist, or a positive allosteric modulator (PAM) of the GABA_{A} receptor. In certain embodiments, the GABA_{A} agent is peripherally restricted, i.e., has limited to no ability to cross the blood-brain barrier, and therefore is substantially restricted to the peripheral nervous system.

In certain embodiments, the methods of evaluating tactile hypersensitivity and/or sensorimotor impairment in a human subject disclosed herein further comprise administering to the subject one or more screening/assessment procedures selected from: demographics and/or family history questionnaire; neurological and physical exam including screening for peripheral neuropathy; medical history questionnaire; medication review; inclusion/exclusion criteria review; pregnancy test and menstrual history questionnaire for females of childbearing potential; Transcranial Magnetic Stimulation (TMS) safety screening; Mini Mental State Evaluation (MMSE); verbal and/or non-verbal intelligence testing; Brain-derived neurotrophic factor (BDNF), Catechol-O-methyltransferase (COMT), and/or apolipoprotein E (APOE) genotyping; Autism Diagnostic Observation Schedule (ADOS); Autism Spectrum Quotient (AQ); Pupil dilation; and Eyes Test.

In certain embodiments, the methods of evaluating tactile hypersensitivity and/or sensorimotor impairment in a human subject disclosed herein further comprise administering to the subject an acoustic prepulse inhibition assay (APPI).

In certain embodiments, the neuro-typical controls in the methods of evaluating tactile hypersensitivity and/or sensorimotor impairment in a human subject disclosed herein are age-matched, gender-matched, and/or IQ-matched. In certain embodiments, the neuro-typical controls have no history of ASD or other developmental delay in themselves or in any known first-degree relatives.

In certain embodiments, the human subject in the methods of evaluating tactile hypersensitivity and/or sensorimotor impairment in a human subject disclosed herein is subjects 18-65 years of age.

In certain embodiments, the subject in the methods of evaluating tactile hypersensitivity and/or sensorimotor impairment in a human subject disclosed herein has been clinically diagnosed with a disorder on the ASD spectrum according to the Diagnostic and Statistical Manual of Mental Disorders (DSM), which appears in various versions (e.g., DSM-IV or DSM-5). In certain embodiments, the subject in the methods of evaluating tactile hypersensitivity and/or sensorimotor impairment in a human subject disclosed herein has been clinically diagnosed with a disorder on the ASD spectrum according to the DSM-IV or DSM-5.

In certain embodiments, an assay result indicating increased tactile hypersensitivity and/or sensorimotor impairment relative to neuro-typical controls in the methods of evaluating tactile hypersensitivity and/or sensorimotor impairment in a human subject disclosed herein, indicates the presence of a disorder in the subject, wherein the disease or disorder is selected from ASD, RTT, PMS, Fragile X syndrome, Neurofibromatosis, and Tuberous Sclerosis complex. In certain embodiments, the degree of tactile hypersensitivity and/or sensorimotor impairment relative to the neuro-typical controls indicates the severity of the disorder in the subject.

In certain embodiments, of the methods of evaluating tactile hypersensitivity and/or sensorimotor impairment in a human subject disclosed herein, the ASD of the subject is syndromic or non-syndromic.

Another aspect of the disclosure, relates to a method for assessing the efficacy of a treatment for a disorder characterized by tactile hypersensitivity and/or sensorimotor impairment comprising: evaluating tactile hypersensitivity and/or sensorimotor impairment in a human subject by any method disclosed herein; administering a treatment, as disclosed herein, to the subject; re-evaluating the subject using any of the TPPI assays disclosed herein; and assessing the efficacy of the treatment by comparing the results of the initial and post-treatment administration evaluation of tactile hypersensitivity and/or sensorimotor impairment.

In certain embodiments, the treatment of method for assessing the efficacy of a treatment for a disorder is a pharmacological treatment. In certain embodiments, the pharmacological treatment comprises administration of a GABA_{A} agent. In certain embodiments, the GABA_{A} agent is peripherally restricted.

In certain embodiments, the disorder in the method for assessing the efficacy of a treatment for a disorder is selected from ASD, RTT, PMS, Fragile X syndrome, Neurofibromatosis, and Tuberous Sclerosis complex.

In another aspect, provided herein is a system or apparatus for performing an assay a method disclosed herein, the system or apparatus comprising:

(a) means for administering a tactile prepulse to a human subject;

(b) means for administering a startle stimulus to the subject; and

(c) means for measuring the subject's response to the startle stimulus.

### BRIEF DESCRIPTION OF THE FIGURES

FIG. 1A: shows percent inhibition of the startle response to a 120-dB noise, when the startle stimulus is preceded by a light air puff of varying intensity in neurotypical controls. One-sample t-test, **, p<0.01; ***, p<0.005. One-way ANOVA with post-hoc Tukey's test, #, p<0.01; *, p<0.05. N=7 neurotypical controls. FIG. 1B: shows percent PPI as a function of ISI for 5 human subjects. FIG. 1C: shows percent PPI as a function of ISI.

FIG. 2 shows that compound 5 (3-aminocyclohex-1-ene-1-carboxylic acid) reduces hairy skin hypersensitivity in Mecp2 and Shank3 mutant mice. Tactile PPI performance in Mecp2 or Shank3 conditional mutant mice, and their control littermates, 30 minutes following administration of either saline or 2 mg/kg compound 5.

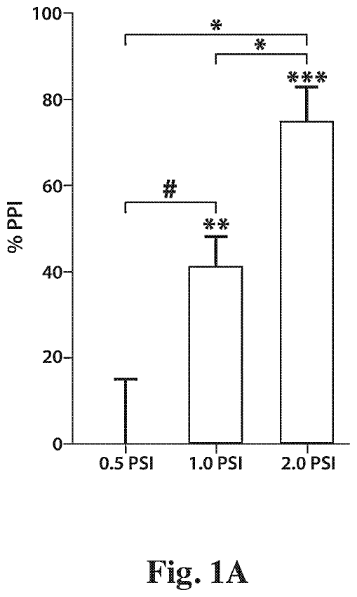

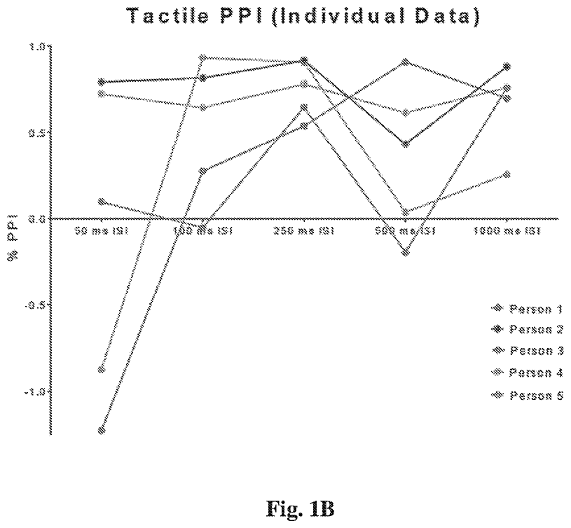

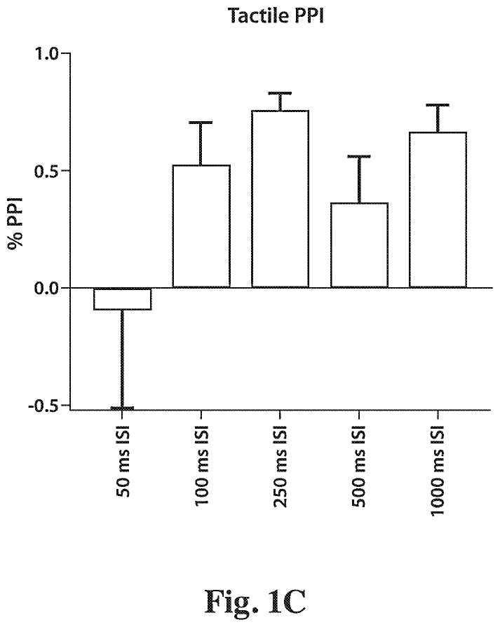

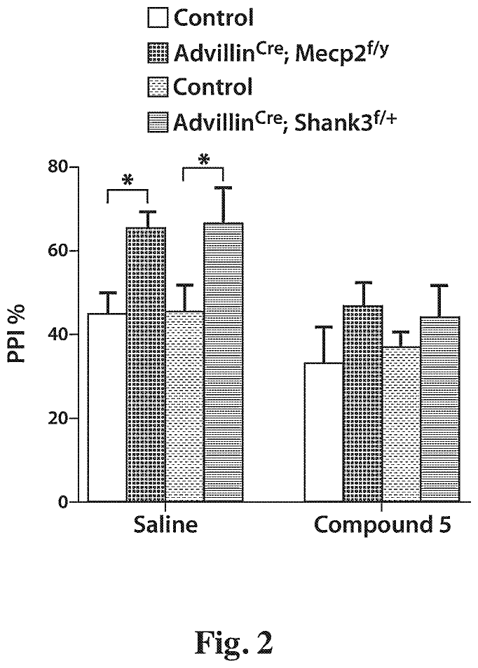

### DETAILED DESCRIPTION

Individuals with Autism spectrum disorders (ASD) commonly experience tactile hypersensitivity. The degree of this somatosensory impairment correlates with increased anxiety behaviors and impairment in social behavior among individuals with ASD. To date, there remains an unmet need for treating tactile hypersensitivity, which may improve anxiety and other core symptoms of ASD. Methodologies and metrics for measuring tactile sensitivity vary widely across clinical and basic research fields. As compared with behavioral methods, methods of evaluating tactile hypersensitivity, or somatosensory impairment in general, based on empirical measurements offer inherent advantages due to the lack of variability in subjective response of subject. There is an urgent need for direct and objective sensory reactivity metrics in clinical studies to assess deficits in specific patient populations and for designing effective therapeutic strategies.

The present disclosure provides a new application of the phenomenon of prepulse inhibition (PPI). PPI is the neurological phenomenon, whereby the application of a first stimulus weakens the neurological reaction of a subject to a second stimulus. Often, the first stimulus is weaker than that of the second stimulus. This reduction in reaction is thought to be caused by the adaptation of the neurological system to adapt to sensory stimuli.

Accordingly, one aspect of the present disclosure provides a tactile prepulse inhibition (TPPI) assay, which comprises the steps of: (a) administering a tactile prepulse to a subject; (b) administering a startle stimulus to the subject; and (c) measuring the subject's response to the startle stimulus. A TPPI assay allows for quantitative measurement of tactile sensitivity without relying on a subject's perception and rating scale, which may be problematic due to the subjective nature of subject interpretation, which may be further exacerbated in ASD individuals where speech and cognitive deficits are common.

A prepulse, as used in a general context, is defined as first stimulus (e.g., auditory, tactile, visual, gustatory, olfactory) to a subject, which stimulates the neurological system of the subject, which first stimulus inhibits the neurological reaction of a subsequent second stimulus (e.g., auditory, tactile, visual, gustatory, olfactory). In certain embodiments, the prepulse may be any stimulus which causes a neurological response in a subject through one of the subjects senses (i.e., auditory, tactile, visual, gustatory, olfactory). In certain embodiments, the prepulse may be a combination of stimuli (e.g., auditory and visual, auditory and tactile). For example, in certain embodiments, more than one selected from, auditory, tactile, visual, gustatory, olfactory, or a combination thereof. In certain embodiments, the prepulse may auditory or tactile. In a particular embodiment, the prepulse is auditory. In another particular embodiment, the prepulse is tactile.

Any means of eliciting a neurological response in a subject by means of exposure to an auditory cue (e.g., sound) may be used. For example, and without being bound by example, a tone produced by an instrument, clanging of objects, electrical or computer generated or reproduced means (e.g., white noise, radio signal), vocal noises, or other sound generation means, may be employed. In certain embodiments, the prepulse generated electronically and is white noise. In some In certain embodiments, the prepulse may be tactile. Any means of eliciting a neurological response in a subject by means of exposure to a tactile cue (e.g., touch, pressure by solid, liquid, gas) may be used. For example, and without being bound by example, an air puff (e.g., gas), touch from another subject or human, touch from a non-human, application of an object by another subject, human, or by application of an object by mechanical means, may be used. In certain embodiments, the tactile prepulse may be an air puff.

An air puff, as used herein, refers to the intentional exposure of the subject to a gas sufficient to elicit a neurological reaction. The gas, while referred to as air, does not need to be the same composition of atmospheric air, but may be any suitable substitute (e.g., oxygen, nitrogen), one of skill in the art is able to select an appropriate gas for use as an air puff. In certain embodiments, the air puff is comprised of a gas selected from, oxygen, air, nitrogen, carbon dioxide, or a combination thereof.

The tactile prepulse may also be directed or concentrated to a particular portion of the subject's anatomy (e.g., arm, hand, palm, thigh, leg, foot, neck). A person of skill in the art will readily able to determine suitable areas for application of a tactile prepulse, but any area capable of eliciting a neurological response in a subject may be used. In certain embodiments, the tactile prepulse is directed at the subject's forearm, the hairy skin on the back of the subject's hand, or the glabrous skin on the palm of the subject's hand. In certain embodiments, the tactile prepulse is directed at the subject's forearm. In certain embodiments, the tactile prepulse is directed at the hairy skin on the back of the subject's hand. In certain embodiments, the tactile prepulse is directed at the glabrous skin on the palm of the subject's hand. In certain embodiments, the tactile prepulse is directed at the subject's thigh, leg, foot, or neck. In certain embodiments, the tactile prepulse is directed at the subject's thigh. In certain embodiments, the tactile prepulse is directed at the subject's leg. In certain embodiments, the tactile prepulse is directed at the subject's foot. In certain embodiments, the tactile prepulse is directed at the subject's neck.

When a tactile prepulse is used, the pressure exerted on the subject by the tactile prepulse may be varied (e.g., less or more pressure applied to the subject). In certain embodiments, the tactile prepulse is applied with a pressure selected from greater than or equal to about 0.1 PSI to less than or equal to about 10 PSI. In certain embodiments, the tactile prepulse is applied with a pressure of about 0.9 PSI to about 3 PSI. In certain embodiments, the tactile prepulse is applied with a pressure selected from 0.1 PSI, 0.2 PSI, 0.3 PSI, 0.4 PSI, 0.5 PSI, 0.6 PSI, 0.7 PSI, 0.8 PSI, 0.9 PSI, 1 PSI, 1.1 PSI, 1.2 PSI, 1.3 PSI, 1.4 PSI, 1.5 PSI, 1.6 PSI, 1.7 PSI, 1.8 PSI, 1.9 PSI, 2 PSI, 2.1 PSI, 2.2 PSI, 2.3 PSI, 2.4 PSI, 2.5 PSI, 2.6 PSI, 2.7 PSI, 2.8 PSI, 2.9 PSI, 3 PSI, 3.1 PSI, 3.2 PSI, 3.3 PSI, 3.4 PSI, 3.5 PSI, 3.6 PSI, 3.7 PSI, 3.8 PSI, 3.9 PSI, 4 PSI, 4.1 PSI, 4.2 PSI, 4.3 PSI, 4.4 PSI, 4.5 PSI, 4.6 PSI, 4.7 PSI, 4.8 PSI, 4.9 PSI, 5 PSI, 5.1 PSI, 5.2 PSI, 5.3 PSI, 5.4 PSI, 5.5 PSI, 5.6 PSI, 5.7 PSI, 5.8 PSI, 5.9 PSI, 6 PSI, 6.1 PSI, 6.2 PSI, 6.3 PSI, 6.4 PSI, 6.5 PSI, 6.6 PSI, 6.7 PSI, 6.8 PSI, 6.9 PSI, 7 PSI, 7.1 PSI, 7.2 PSI, 7.3 PSI, 7.4 PSI, 7.5 PSI, 7.6 PSI, 7.7 PSI, 7.8 PSI, 7.9 PSI, 8 PSI, 8.1 PSI, 8.2 PSI, 8.3 PSI, 8.4 PSI, 8.5 PSI, 8.6 PSI, 8.7 PSI, 8.8 PSI, 8.9 PSI, 9 PSI, 9.1 PSI, 9.2 PSI, 9.3 PSI, 9.4 PSI, 9.5 PSI, 9.6 PSI, 9.7 PSI, 9.8 PSI, 9.9 PSI, 10 PSI, or any increment therein. In certain particular embodiments, the tactile prepulse is applied with a pressure of about 0.9 PSI. In other particular embodiments, the tactile prepulse is applied with a pressure of about 2 PSI. In certain embodiments, the tactile prepulse is applied with a pressure of about 3 PSI.

In certain embodiments, the duration of the prepulse is 1-5000 milliseconds, e.g., 1-1000, 100-1000, 100-500, or 500-1000 milliseconds.

Subsequent to the exposure to the prepulse, the subject is exposed to a second stimulus is referred to herein as the startle stimulus. The second stimulus (startle stimulus), like the prepulse (first stimulus), is a stimulus which elicits a neurological response in the subject. A startle stimulus, as used in a general context, is defined as second stimulus (e.g., auditory, tactile, visual, gustatory, olfactory) to a subject, which stimulates the neurological system of the subject, subsequent to the prepulse (first stimulus) (e.g., auditory, tactile, visual, gustatory, olfactory). In certain embodiments, the startle stimulus may be any stimulus which causes a neurological response in a subject through one of the subjects senses (i.e., auditory, tactile, visual, gustatory, olfactory). In certain embodiments, the startle stimulus may be a combination of stimuli (e.g., auditory and visual, auditory and tactile). For example, in certain embodiments, more than one stimuli are selected from, auditory, tactile, visual, gustatory, olfactory, or a combination thereof. In certain embodiments, the startle stimulus may be auditory or tactile. In another particular embodiment, the startle stimulus is tactile. The means of the second stimulus, by which a neurological response is elicited, may be auditory (startle stimulus). Any means of eliciting a neurological response in a subject by means of exposure to an auditory cue (e.g., sound) may be used. For example, and without being bound by example, a tone produced by an instrument, clanging of objects, electrical or computer generated or reproduced means (e.g., white noise, radio signal), vocal noises, or other sound generation means, may be employed. In certain embodiments, the prepulse generated electronically and is white noise.

The intensity of the startle stimulus must be sufficient to elicit a neurological response but less than an intensity which would be deleterious or harmful to the subject, and preferably should elicit a greater response than that of the prepulse (when each are measured independently). A person of skill in the art is able to readily assess and determine appropriate intensity levels depending on the startle stimulus selected. In certain embodiments, the startle stimulus has an intensity selected from greater than or equal to about 100 dB to less than or equal to about 150 dB. In certain embodiments, the startle stimulus has an intensity selected from greater than or equal to about 110 dB to less than or equal to about 120 dB. In certain embodiments, the startle stimulus has an intensity selected from 111 dB, 112 dB, 113 dB, 114 dB, 115 dB, 116 dB, 117 dB, 118 dB, 119 dB, and 120 dB. In certain embodiments, the startle stimulus has an intensity of about 115 dB.

The response to the startle stimulus, and the effect of prepulses thereon, is measured at the time of the startle stimulus. Any objective measure which can be reliable coordinate with the startle response may be used, for example EMG, eye-blink reflex, autonomic response, pupillometry, or galvanic skin resistance. EMG is a method of capturing subject responses by capturing and measuring the electrical signals transmitted by motor neurons which cause muscles to contract. An EMG uses electrodes in the subject muscles to translate these signals into graphs, sounds or numerical values. The EMG electrodes are placed in a manner consistent with capturing the target motor neuron signals. One of skill in the art will readily be able to select the placement in contemplation of the target motor neurons. In some embodiments, the EMG electrodes are placed over the ocular muscles, trapezius muscles, scalp, or a combination thereof. In some embodiments, the EMG electrodes are placed over the ocular muscles. In some embodiments, the EMG electrodes are placed over the trapezius muscles. In some embodiments, the EMG electrodes are placed over the scalp. Another method of measuring the neurological response to the startle stimulus is the eye-blink reflex test, which measures the responsiveness of the eyelid of the subject to blink or twitch in response to a stimulus, this is often accomplished with the aid of a photoelectric cell aimed at the eyelid to capture the movement of the eyelid. A further method by which the neurological response to the startle stimulus can be measured is by measuring the autonomic response of the subject. Autonomic responses are related to any of a large number of normal reflexes governing and regulating the functions of the viscera. Autonomic reflexes control such activities of the body as blood pressure, heart rate, peristalsis, sweating, and urination and autonomic response measurements capture how the systems in the body controlled by the autonomic nerves respond to stimulation. The data collected during testing will indicate if the autonomic nervous system has been stimulated. A further method by which the neurological response to the startle stimulus can be measured is by pupillometry. Pupillometry is a method of measuring response in a subject by assessment, either manually or automatically (e.g., mechanically, automation, or by digital/computer means, pupilometer) of the pupil reaction in the subject's eye. A further method by which the neurological response to the startle stimulus can be measured is by measuring the galvanic response of the subject. Galvanic skin resistance, also known as the electro-dermal response, is the phenomenon that the skin has continuous variation in the electrical characteristics of the skin and momentarily becomes a better conductor of electricity when either external or internal stimuli occur that are neurologically arousing. Galvanic skin resistance has been found to be a strong predictor of attention and memory. In certain embodiments, the subject's response to the startle stimulus is measured by EMG. In certain embodiments, the subject's response to the startle stimulus is measured by eye-blink reflex. In certain embodiments, the subject's response to the startle stimulus is measured by the subject's autonomic response. In certain embodiments, the subject's response to the startle stimulus is measured by pupillometry. In certain embodiments, the subject's response to the startle stimulus is measured by galvanic skin resistance.

The startle stimulus is administered at some period of time subsequent to the prepulse, the duration between the administration of the prepulse and the startle stimulus is referred to herein as the “ISI.” The ISI influences the effect of the prepulse on the response observed from the startle stimulus. For example, some studies have shown that a shorter ISI decreases the observed reaction to the startle stimulus, as compared to longer ISI periods. Typical ISI periods range from as short as about 5 ms to as long as about 1500 ms. In certain embodiments, the ISI is selected from greater than or equal to about 15 ms to less than or equal to about 1000 ms. In certain embodiments, the ISI is selected from greater than or equal to about 150 ms to less than or equal to about 350 ms. In certain embodiments, the ISI is selected from 150 ms, 155 ms, 160 ms, 165 ms, 170 ms, 175 ms, 180 ms, 185 ms, 190 ms, 195 ms, 200 ms, 205 ms, 210 ms, 215 ms, 220 ms, 225 ms, 230 ms, 235 ms, 240 ms, 245 ms, 250 ms, 255 ms 260 ms, 265 ms, 270 ms, 275 ms, 280 ms, 285 ms, 290 ms, 295 ms, 300 ms, 305 ms, 310 ms, 315 ms, 320 ms, 325 ms, 330 ms, 335 ms, 340 ms, 345 ms, and 350 ms. In certain embodiments, the ISI is about 250 ms.

To isolate and reduce the possibility of external or environmental stimuli interfering or having an effect on the subject's response to the TPPI assay (i.e., the prepulse, startle stimulus, and the effect of each on neurological response described herein), the subject may be isolated or otherwise insulated from stimuli (e.g., auditory, visual, tactile), either before or during the TPPI assay. For example, a subject may wear noise-cancelling headphones, either prior or during the TPPI assay. In certain embodiments, the subject is wearing noise-cancelling headphones prior to the TPPI assay. In certain embodiments, the subject is wearing noise-cancelling headphones during the TPPI assay. In certain embodiments, the subject is wearing noise-cancelling headphones prior to and during the TPPI assay.

In another aspect, the disclosure relates to a method of evaluating the tactile hypersensitivity and or sensorimotor impairment in a human subject comprising: administering a TPPI assay as disclosed herein; comparing the assay results to neuro-typical controls; and determining the degree of tactile hypersensitivity. In certain embodiments, data regarding neuro-typical controls may be obtained from scientific literature. In other embodiments, neuro-typical control data is generated as described herein. In certain embodiments, neuro-typical controls are subjects who do not have any developmental disabilities (e.g., ASD, are not on the ASD spectrum), developmental coordination disorder, or attention deficit hyperactivity disorder. In certain embodiments, the neuro-typical controls are selected at the same time as the subject. In certain embodiments, the neuro-typical controls are previously selected, and in certain embodiments, the neuro-typical controls are results from testing of neuro-typical controls previously selected and which have previously completed the TPPI assay. In certain embodiments, the neuro-typical controls are subsequently selected, and in certain embodiments, the neuro-typical controls are results from testing of neuro-typical controls subsequently selected and which complete, or will complete the TPPI assay after the subject. Neuro-typical controls may also be matched to the subject in various demographic ways (e.g., age, gender, intelligence quotient (IQ), biology). In certain embodiments, the neuro-typical controls are age-matched, gender-matched, IQ-matched, or a combination thereof. In certain embodiments, the neuro-typical controls are age-matched. In certain embodiments, the neuro-typical controls are gender-matched. In certain embodiments, the neuro-typical controls are IQ-matched. Further, neuro-typical controls may also be selected based on family history of any biological or neurological criteria. In certain embodiments, the neuro-typical controls have no history of ASD or other developmental delay in themselves or in any known first-degree relatives.

The degree of tactile hypersensitivity, as used herein, refers to the assessing difference between the neurological responses of the subject and the neuro-typical controls. The differences will depend on the modality of measurement used, but one of ordinary skill in the art will immediately appreciate the methods and means by which to read the measurements and assess their meaning.

In some instances, the results may indicate effectiveness or ineffectiveness, or a degree therein of a particular treatment. Accordingly, the results may be interpreted to indicate the severity of the disorder, and the effect or non-effect that treatment has on a subject. In certain embodiments, the degree of tactile hypersensitivity and/or sensorimotor impairment indicates the severity of the disorder in the subject.

In certain embodiments, the method further comprises adjusting a treatment of tactile hypersensitivity and/or sensorimotor impairment in the subject. Adjusting a treatment may comprise changing the dosage of a pharmacological treatment, e.g., increasing or decreasing the dosage, or changing the pharmacological agent being administered, e.g., switching from a GABA_{A} agonist to a GABA reuptake inhibitor, or switching from a small molecule therapeutic to a biological therapeutic. Adjusting a treatment may also comprise increasing or decreasing the duration of the treatment.

The subject of any of the methods herein may be selected from any individual who needs, or is suspected of needing, evaluation for sensory hypersensitivity. The subjects may be of any age, gender, race, ethnicity, or other demographic metric. In certain embodiments, the age of the subject is between about 18 to about 65. In certain embodiments, the subject is an adult. In certain embodiments, the subject is an adolescent. The adolescent may be 13-15 years old, 14-16 years old, 15-17 years old, 16-18, years old, or 17-19 years old.

In certain embodiments, the subject is a child. In certain embodiments, the child is less than 12 years of age. In certain embodiments, the child is less than 10 years of age. In certain embodiments, the child is less than 8 years of age. In certain embodiments, the child is less than 6 years of age. In certain embodiments, the child is less than 4 years of age. In certain embodiments, the child is less than 2 years of age. In certain embodiments, the child is 2-4 years of age. In certain embodiments, the child is 4-6 years of age. In certain embodiments, the child is 6-8 years of age. In certain embodiments, the child is 8-10 years of age. In certain embodiments, the child is greater than 12 years of age.

The subject may have cognitive defects, disease states, disorders (e.g., syndrome, disorder (such as ASD), complex), or an individual for whom treatment for any such indication is contemplated. Evaluation for one of these criteria may be done in any validated manner (e.g., evaluation by a professional in the area of interest), for example by clinical diagnosis. Various tools exist to complete an assessment of subjects for mental disorders, for example the DSM. The DSM is considered to be the authoritative guide used by healthcare professionals (e.g., doctors, professionals in the area of interest) throughout the world, and particularly in the United States of America, for the diagnosis of mental disorders. It has gone through various revisions and appears in a variety of versions, two of which are recent iterations, the DSM-IV and DSM-5. In certain embodiments, the subject has, or is suspected of having one or more of ASD, RTT, PMS, Fragile X syndrome, Neurofibromatosis, or Tuberous Sclerosis complex. In certain embodiments, the subject has ASD, or is suspected of having ASD. In certain embodiments, the subject has, or is suspected of having RTT. In certain embodiments, the subject has, or is suspected of having PMS. In certain embodiments, the subject has, or is suspected of having Fragile X syndrome. In certain embodiments, the subject has, or is suspected of having Neurofibromatosis. In certain embodiments, the subject has, or is suspected of having Tuberous Sclerosis complex. In certain embodiments, the subject having, or suspected of having, a mental disorder has been diagnosed according the DSM-IV or DSM-5. In certain embodiments, a subject having, or suspected of having ASD, or being on the ASD spectrum, has been diagnosed according to the DSM-IV or DSM-5.

A treatment as used herein, refers to a course of action or management of the care of the subject for the modulation or amelioration of a particular state experienced by the subject. For example, treatments may be forms of cognitive therapy (e.g., counseling, psychotherapy), physiological (e.g., manual manipulation, exercise, applied stimuli) therapy, or pharmacological (e.g., biologics, chemical, placebo, diet) therapy, or a combination thereof. Pharmacological treatments can be, any compound administered to the subject to effectuate treatment (e.g., prescription compounds, over-the-counter (OTC) compounds, foods, placebo). In certain embodiments, the pharmacological treatment of the disorder comprises administration of a small molecule drug, a large molecule drug (e.g., an oligosaccharide), or a biologic drug (e.g., a peptide, antibody, or oligonucleotide) to the subject. In certain embodiments, the pharmacological treatment comprises the administration of a GABA_{A} agent (e.g., a GABA reuptake inhibitor or transport inhibitor, a GABA_{A} receptor agonist, or a positive allosteric modulator (PAM) of the GABA_{A} receptor.

Representative GABA agents are disclosed in U.S. Provisional Application No. 62/674,770, filed on May 22, 2018, 62/823,360, filed on Mar. 25, 2019, and 62/677,367, filed May 29, 2018, 62/823,419, filed Mar. 25, 2019, and 62/823,391, filed Mar. 25, 2019, each of which is incorporated herein by reference in their entirety.

In certain embodiments, the GABA_{A} agent is peripherally restricted, i.e., has limited to no ability to cross the blood-brain barrier, and therefore is substantially restricted to the peripheral nervous system. The GABA_{A} receptor is an ionotropic ligand-gated ion channel receptor in the central nervous system. GABA_{A} receptor is believe to play a role in a wide variety of neurological disorders (e.g., ASD). However, in many instances GABA_{A} ligands can have side effects. Currently, GABA_{A} ligands penetrate the brain and cause sedation as well as cognitive issues such as sedation and complications associated with abnormal brain development. Accordingly, restricting the treatment to peripheral systems (e.g., not treating across the blood brain barrier) is preferred. In certain embodiments, the subject is treated using any form of treatment intended to modulate their disorder. In certain embodiments, the subject is treated using any form of treatment intended to improve their disorder.

The method may also include using additional information to inform the modalities of prepulse, modality of measurement, interpreting of the results of the tests, or matching of the subjects to appropriate neuro-typical controls. In certain embodiments, one or more screening/assessments may be used: demographics and/or family history questionnaire; neurological and physical exam including screening for peripheral neuropathy; medical history questionnaire; medication review; inclusion/exclusion criteria review; pregnancy test and menstrual history questionnaire for females of childbearing potential; Transcranial Magnetic Stimulation (TMS) safety screening; Mini Mental State Evaluation (MMSE); verbal and/or non-verbal intelligence testing; Brain-derived neurotrophic factor (BDNF), Catechol-O-methyltransferase (COMT), and/or apolipoprotein E (APOE) genotyping; Autism Diagnostic Observation Schedule (ADOS); Autism Spectrum Quotient (AQ); Pupil dilation; and Eyes Test.

Additionally, more than one PPI assay, as described herein, may be performed on a subject. The tests can be run sequentially (i.e., one followed by a second or any number of tests), or concurrently with the prepulse stimuli overlapping. The tests may further have different prepulse stimuli (e.g., acoustic and tactile) and/or measuring modalities (e.g., eye-blink, pupillometry). In certain embodiments, a TPPI assay as described herein comprises a second PPI assay where one of the TPPI assays comprises a tactile prepulse and the other PPI assay comprises an acoustic prepulse (APPI).

In certain embodiments, the subject has, or is suspected of having syndromic ASD. In certain embodiments, the subject has, or is suspected of having non-syndromic ASD.

Another aspect of the disclosure relates to a method of assessing the efficacy of a treatment for a disorder, which disorder is characterized by tactile hypersensitivity and/or sensorimotor impairment comprising: administering to the subject a TPPI assay according to any one of the methods disclosed herein; administering a treatment to the subject; comparing the assay results to neuro-typical controls; and determining the degree of tactile hypersensitivity and/or sensorimotor impairment in the subject.

In certain embodiments, the subject is treated using any form of treatment intended to modulate their disorder. In certain embodiments, the subject is treated using any form of treatment intended to improve their disorder. In certain embodiments, the form of treatment is pharmacological. In some embodiment, the pharmacological treatment is a GABAA agent. In some embodiment, the GABAA agent is peripherally restricted.

In certain embodiments, the subject is selected from any of those described herein. In certain embodiments, the subject has, or is suspected of having one or more of ASD, RTT, PMS, Fragile X syndrome, Neurofibromatosis, or Tuberous Sclerosis complex. In certain embodiments, the subject has ASD, or is suspected of having ASD. In certain embodiments, the subject has, or is suspected of having RTT. In certain embodiments, the subject has, or is suspected of having PMS. In certain embodiments, the subject has, or is suspected of having Fragile X syndrome. In certain embodiments, the subject has, or is suspected of having Neurofibromatosis. In certain embodiments, the subject has, or is suspected of having Tuberous Sclerosis complex.

### Indications

The assays described herein are useful for diagnosing and evaluating the extent of tactile dysfunction, anxiety, and social impairment in a subject diagnosed with ASD, RTT, PMS, or Fragile X syndrome.

### Tactile Dysfunction

Tactile dysfunction includes exhibiting symptoms such as withdrawing when being touched, refusing to eat certain “textured” foods and/or to wear certain types of clothing, complaining about having hair or face washed, avoiding getting hands dirty (e.g., glue, sand, mud, finger-paint), and using finger tips rather than whole hands to manipulate objects. Tactile dysfunction may lead to a misperception of touch and/or pain (hyper- or hypo-sensitive) and may lead to self-imposed isolation, general irritability, distractibility, and hyperactivity.

### Anxiety

Anxiety includes emotions characterized by feelings of tension, worried thoughts and physical changes like increased blood pressure. Anxiety can be characterized by having recurring intrusive thoughts or concerns, avoiding certain situations (e.g., social situations) out of worry, and physical symptoms such as sweating, trembling, dizziness or a rapid heartbeat.

### Social Impairment

Social impairment involves a distinct dissociation from and lack of involvement in relations with other people. It can occur with various mental and developmental disorders, such as autism. Social impairment may occur when an individual acts in a less positive way or performs worse when they are around others as compared to when alone. Nonverbal behaviors associated with social impairment can include deficits in eye contact, facial expression, and gestures that are used to help regulate social interaction. Often there is a failure to develop age-appropriate friendships. Social impairment can also include a lack of spontaneous seeking to share achievements or interests with other individuals. A person with social impairment may exhibit a deficit in social reciprocity with individuals, decreased awareness of others, lack of empathy, and lack of awareness of the needs of others.

### Autism Spectrum Disorder

ASD is a heterogeneous group of neurodevelopmental disorders as classified in the fifth revision of the American Psychiatric Association's Diagnostic and Statistical Manual of Mental Disorders 5^{th} edition (DSM-5). The DSM-5 redefined the autism spectrum to encompass the prior (DSM-IV-TR) diagnosis of autism, Asperger syndrome, pervasive developmental disorder not otherwise specified, childhood disintegrative disorder, and Rett syndrome. The autism spectrum disorders are characterized by social deficits and communication difficulties, stereotyped or repetitive behaviors and interests, and in some cases, cognitive delays. For example, an ASD is defined in the DSM-5 as exhibiting (i) deficits in social communication and interaction not caused by general developmental delays (must exhibit three criteria including deficits in social-emotional reciprocity, deficits in nonverbal communication, and deficits in creating and maintaining relationships appropriate to developmental level), (ii) demonstration of restricted and repetitive patterns of behavior, interest or activities (must exhibit two of the following four criteria: repetitive speech, repetitive motor movements or repetitive use of objects, adherence to routines, ritualized patterns of verbal or nonverbal, or strong resistance to change, fixated interests that are abnormally intense of focus, and over or under reactivity to sensory input or abnormal interest in sensory aspects of environment), (iii) symptoms must be present in early childhood, and (iv) symptoms collectively limit and hinder everyday functioning. ASD is also contemplated herein to include Dravet's syndrome and autistic-like behavior in non-human animals.

### Rett Syndrome

Rett syndrome is an X-linked disorder that affects approximately one in ten-thousand girls. Patients go through four stages: Stage I) Following a period of apparently normal development from birth, the child begins to display social and communication deficits, similar to those seen in other autism spectrum disorders, between six and eighteen months of age. The child shows delays in their developmental milestones, particularly for motor ability, such as sitting and crawling. Stage II) Beginning between one and four years of age, the child goes through a period of regression in which they lose speech and motor abilities, developing stereotypical midline hand movements and gait impairments. Breathing irregularities, including apnea and hyperventilation also develop during this stage. Autistic symptoms are still prevalent at this stage. Stage III) Between age two and ten, the period of regression ends and symptoms plateau. Social and communication skills may show small improvements during this plateau period, which may last for most of the patients' lives. Stage IV) Motor ability and muscle deterioration continues. Many girls develop severe scoliosis and lose the ability to walk.

### Phelan McDermid Syndrome

Phelan McDermid syndrome is a rare genetic condition caused by a deletion or other structural change of the terminal end of chromosome 22 in the 22q13 region or a disease-causing mutation of the Shank3 gene. Although the range and severity of symptoms may vary, PMS is generally thought to be characterized by neonatal hypotonia (low muscle tone in the newborn), normal growth, absent to severely delayed speech, moderate to profound developmental delay, and minor dysmorphic features. People who have PMS often show symptoms in very early childhood, sometimes at birth and within the first six months of life.

### Fragile X Syndrome

Fragile X syndrome is an X chromosome-linked condition that is characterized by a visible constriction near the end of the X chromosome, at locus q27.3 that causes intellectual disability, behavioral and learning challenges and various physical characteristics Fragile X syndrome is the most common inherited form of mental retardation and developmental disability. Males with Fragile X syndrome usually have mental retardation and often exhibit characteristic physical features and behavior. Fragile X syndrome is characterized by behavior similar to autism and attention deficit disorder, obsessive-compulsive tendencies, hyperactivity, slow development of motor skills and anxiety fear disorder. When these disabilities are severe and occur simultaneously, the condition is sometimes described as autism, and may be associated with any degree of intelligence. Other characteristics are a likable, happy, friendly personality with a limited number of autistic-like features such as hand-flapping, finding direct eye contact unpleasant, and some speech and language problems. Physical features may include large ears, long face, soft skin and large testicles (called “macroorchidism”) in post-pubertal males. Connective tissue problems may include ear infections, flat feet, high arched palate, double-jointed fingers and hyper-flexible joints.

### Neurofibromatosis

Neurofibromatosis is a genetic disorder that causes tumors to form on nerve tissue. These tumors can develop anywhere in your nervous system, including your brain, spinal cord and nerves. Neurofibromatosis is usually diagnosed in childhood or early adulthood. The tumors are usually noncancerous (benign), but sometimes can become cancerous (malignant). Symptoms are often mild. However, complications of neurofibromatosis can include hearing loss, learning impairment, heart and blood vessel (cardiovascular) problems, loss of vision, and severe pain.

### Tuberous Sclerosis Complex

Tuberous sclerosis complex is a genetic disorder characterized by the growth of numerous noncancerous (benign) tumors in many parts of the body. These tumors can occur in the skin, brain, kidneys, and other organs, in some cases leading to significant health problems. Tuberous sclerosis complex also causes developmental problems, and the signs and symptoms of the condition vary from person to person.

### EXAMPLES

### Example 1

A range of mouse genetic models of ASD is combined with behavioral testing, synaptic analyses, and electrophysiology to define both the etiology of aberrant tactile sensitivity in ASD and the contribution of somatosensory dysfunction to the expression of ASD-like traits (Orefice et al., Cell, 2016; Orefice et al., 2019, Cell 178, 867-886; Tasnim et al., unpublished). It was found that mutations in genes associated with both syndromic and non-syndromic forms of ASD cause tactile dysfunction, and that the RTT-, Phelan McDermid- and autism-associated genes Mecp2, Shank3, and Gabrb3 function cell autonomously in peripheral somatosensory neurons for normal tactile behaviors. Tactile dysfunction associated with Mecp2 and Gabrb3 ASD models is caused by a deficiency of the β3 subunit of the GABAA receptor (GABRB3) and GABAA receptor-mediated presynaptic inhibition (PSI) of somatosensory inputs to the CNS. Shank3 mutant DRG neurons, on the other hand, exhibit hypersensitivity (Orefice et al., 2019, Cell 178, 867-886). These somatosensory deficits during development contribute to aberrant brain development, and social behaviors, including anxiety-like behaviors and social impairments, in adulthood. These findings indicate that somatosensory neuron dysfunction causes tactile processing deficiency and over-reactivity, and that these deficiencies during development lead to alterations in cortical microcircuit function, anxiety-like behavior and social interaction deficits in adult mice.

GABA_{A} receptor agonist isoguanine, which acts directly on GABA_{A} receptors on peripheral mechanosensory neurons to attenuate their activity, was shown to be useful to reduce tactile over-reactivity as well as anxiety and social impairments in a range of ASD mouse models (Orefice et al., 2019, Cell 178, 867-886). A key consideration, is that treating young children with GABA_{A} receptor agonists and positive allosteric modulators (PAMs) has traditionally been avoided because of undesirable side effects of these drugs in children. It was investigated to develop novel peripherally-restricted GABA_{A} receptor agonists, PAMs, and GABA reuptake inhibitors, i.e. those that do not cross the blood-brain barrier, to treat tactile over-reactivity and core ASD behaviors. Such peripherally-restricted compounds should not produce undesirable side effects observed with all currently used, FDA-approved GABA_{A} receptor drugs that act in the brain.

Somatosensory sensitivity and processing deficits are investigated in both children and adults, including those diagnosed with idiopathic ASD, as well as genetic/syndromic forms including Rett Syndrome (Mecp2), Fragile X Syndrome (Fmr1), Phelan MacDermid Syndrome (Shank3), Neurofibromatosis (NF1) and Tuberous Sclerosis complex (Tsc1/2), to assess phenotype convergence and measure the pervasiveness of somatosensory abnormalities across forms of ASD during both development and adulthood. The findings allow differences in somatosensory behaviors in both non-syndromic and syndromic ASD patient populations to be established, and enable direct clinical translation of data to identify the most reliable biomarkers for ASD-associated somatosensory assessment in ASD patients.

A large number of patients in each of the five ASD groups, as well as control subjects, are enrolled to specifically assess cortical plasticity by TMS and related measures. Patients included both children (aged 2-17 years) and adult patients. Participants in each ASD group (groups: non-syndromic ASD, Rett Syndrome, Fragile X Syndrome, Phelan-MacDermid Syndrome, and Tuberous Sclerosis complex) as well as healthy age-matched control subjects for each age group are used. Physiological readouts (tactile PPI, pupil dilation, and EEG) in conjunction with psychophysical perception measures were used.

### Tactile PPI, EEG, Pupil Dilation, and Perceptual-Based Measurements of Tactile Reactivity in ASD Patients

It is first necessary to assess functional differences in tactile responses in patients using a human tactile PPI assay. TPPI provides a quantitative measure of tactile sensitivity that does not rely on perception, verbal communication or a subjects' rating scale, which may be problematic with ASD individuals with speech and cognitive impairments. Human TPPI utilized startle responses to a loud noise measured by electromyography (EMG) or a photoelectric cell aimed at the eyelid to capture the eye blink reflex. Prepulse stimuli is either a light air puff directed at either forearm, back or hand glabrous skin or a low decibel, non-startling acoustic tone (acoustic PPI), for comparison. This paradigm is also used to measure heart rate, pupil dilation, and galvanic skin resistance during both tactile and acoustic PPI assays performed on separate days. Electroencephalogram (EEG) measurements is also taken during each of these assays. ASD subjects exhibit heightened tactile PPI performance due to increased sensitivity to tactile stimuli, as observed in mouse models. In complementary experiments, perceptual experiences to tactile stimulation of glabrous and hairy skin in the syndromic and non-syndromic ASD patients, compared to control subjects, is measured. A battery of somatosensory stimuli is applied to different regions of hairy and glabrous skin of each patient; these included indentation, textured objects, vibration, and thermal (hot and cold temperatures) stimuli. Subjects are asked to rate their threshold of perception and intensity of the various stimuli, as described. The psychophysical metrics, although potentially complicated by cognitive and communication deficiencies and in ASD patients, provide a valuable comparison to the more quantitative physiological measurements obtained in the EEG, pupil dilation measurements, and tactile PPI experiments.

### Example 2

This was a physiologic study aimed at investigating dysfunction of sensorimotor integration in ASD. This study evaluates tactile prepulse inhibition (PPI) and perceptual-based measurements as biomarkers of sensorimotor integration in adults and children with ASD as compared with their age-, gender-, and IQ-matched neuro-typical controls. Sixty participants with ASD in total (including non-syndromic ASD, Rett Syndrome, Fragile X Syndrome, Phelan-McDermid Syndrome, and Tuberous Sclerosis), as well as up to 60 healthy age-, gender- and IQ-matched control subjects are entered into the study. Besides the screening visit, each subject's participation consisted of two sensory-assessment visits.

### Screening and Baseline Neuropsychological Assessment

On visit 1, informed consent is obtained and each participant undergoes the following screening/assessment procedures:

### All Subjects:

Subject demographics, subject family history
Neurological and physical exam including screening for peripheral neuropathy by a neurologist using standard clinical testing (e.g., pin-prick, touch, vibration)
Medical history and medication review
Inclusion and exclusion criteria review
Mini Mental State Evaluation (MMSE)
Verbal and/or non-verbal intelligence testing with the Abbreviated Stanford-Binet test
A saliva sample was taken for BDNF, COMT, and APOE genotyping (note that lack of gathering the saliva sample will not disqualify the participant from the study and is an optional procedure in the consent)—this sample may be collected at any point during participation in the study

### Autistic Subjects Only:

Autism Diagnostic Observation Schedule (ADOS) (standardized evaluation for autism)

### High-Functioning Autistic Adults and Neurotypical Adults Only:

Autism Spectrum Quotient (AQ)

Eyes Test

In total, the visits last approximately 3 hours. Additional time is offered to autistic subjects to introduce them to the equipment and procedures involved in the TMS visits to ensure that they were given all of the information that they needed to be comfortable and prepared.

There are two sensory assessment visits. The first visit consists of tactile PPI, Texture Discrimination, and Von Frey Fibers. The second visits consists of the acoustic PPI only. If an ASD participant is not able to tolerate the PPI session in the first visit, they are not able to return for the second PPI assessment. Adult subjects undergo the following procedures:
Tactile Pre-pulse inhibition (TPPI) assay
Texture discrimination assay with laser-cut objects. Texture discrimination abilities are assessed through exploration of acrylic cubes with varying degrees of roughness. Through multiple assays, subjects are asked to determine differences between objects and to compare roughness between two objects, using either verbal or non-verbal communication to indicate choices.
Sensory assessment with von Frey fibers (indentation stimuli). The mechanical detection threshold (MDT) to indentation stimuli is assessed using a standardized set of modified von Frey hairs (North Coast Medical, Gilroy, Calif.) exerting forces between 0.25 and 512 millinewtons (mN). Each trial consists of a filament being manually lowered normal to the surface of the skin until contact is made with the skin, and continued force applied smoothly until the filament buckled once. Testing occurs on both the hairy skin of the forearm, as well as the glabrous skin on the palm of the hand. Before each presentation, a verbal prompt is given: “pay attention now,” and after each presentation the subject reported whether or not he/she detected the applied filament

### Methods and Procedures

The Autism Diagnostic Observation Schedule (ADOS) is a standardized instrument for diagnosing and assessing individuals with ASD. It consists of a series of structured and semi-structured tasks that involve social interaction between the examiner and the subject. The examiner observes and identifies segments of the subject's behavior and assigns these to predetermined observational categories. The ADOS generally takes 30-60 min to administer. The examiner provides a series of opportunities for the subject to show social and communication behaviors relevant to the diagnosis of autism. Categorized observations are subsequently combined to produce quantitative scores for analysis. Research-determined cut-offs identify the potential diagnosis of ASD, allowing a standardized assessment of autistic symptoms.

Autism Spectrum Quotient (AQ) is a questionnaire that can also be used as a screening tool for ASD. This questionnaire can be filled out directly by the participant, and is used with the control and high-functioning autistic subjects in the adult population.

Eyes Test is a measure of the capacity to derive emotional expression based only on a photograph of a person's eyes. The photograph has 4 descriptors around the eyes. The participant is asked to circle which of the words best describes what the eyes are expressing. It is also seen as a theory of mind test. It is used with the control and high functioning autistic subjects in the adult population.

Short Sensory Profile (SSP) is a questionnaire use to measure responses to sensory events in daily life. The subject or caregiver completes the Sensory Profile by assessing the frequency of the subject's responses to certain sensory processing, modulation, and behavioral/emotional events as described in 125 different items. This test is administered to both subjects with ASD as well as control subjects.

### Sensory Testing

### Prepulse Inhibition of the Startle Reflex (PPI)

Testing sessions consisted of an acclimation phase, block I, block II, block III and block IV trials. Each subject first underwent an acclimation phase to familiarize the person to the equipment (headphones, stimulator, etc.) and background noise. Subjects were seated in a comfortable chair in a quiet room. Each subject was instructed to sit still, focus their eyes on a spot on the wall in front of them, and stay awake. Subjects were informed that for the next 30 minutes, they would hear “bip-sounds” through the headphones they will where, and some of them is loud. Subjects are constantly observed and instructed to signal if they needed help or wanted to terminate the experiment. Each subject undergoes a 5-minute acclimation period, during which constant background noise of broadband white noise (65 dB) was presented through the headphones. After the 5-minute acclimation phase, trials begin which are superimposed over the background noise.

Block I consists of 5 startle stimuli alone to measure the initial startle reflex. For adult subjects, the startle stimulus was a broadband white noise of 115 dB, and for children the startle stimulus was a 105 dB broadband white noise. The startle stimulus was presented for a duration of 20 ms (instant rise and fall), with randomized inter-trial intervals (ITI) between 10 and 50 seconds, with an average ITI of 30 seconds.

Block II consisted of 5 prepulse stimuli alone. For the acoustic PPI session, prepulse stimuli were a 68, 71, 74, 77, or 80-dB broadband white noise that lasted for 20 ms (instant rise and fall). For the tactile PPI session, a 0.9 PSI air puff was administered to the glabrous skin on the palm of the hand, the hairy skin on the back of the hand, or the hairy skin on the forearm.

Block III incorporated 35 prepulse/pulse, startle stimulus alone, and no stimulation trials which were pseudorandomized. For acoustic PPI, the prepulse was 20 ms in duration and presented 100 ms before the startle stimulus (inter-stimulus interval, ISI). For tactile PPI, the prepulse intensity remained constant (0.9 PSI, 50 ms), and the ISI was be varied from 50 ms to 1 second in duration.

Block IV consisted of 5 startle stimuli alone, to measure habituation to the startle stimuli over the testing session. For adult subjects, the startle stimulus was a broadband white noise of 115 dB, and for children the startle stimulus was a 105 dB broadband white noise. The startle stimulus was presented for a duration of 20 ms (instant rise and fall), with randomized inter-trial intervals (ITI) between 10 and 50 seconds, with an average ITI of 30 seconds.

Throughout the session, startle reflex is measured by electromyography (EMG) activity from the right m. orbicularis oculi, or a photoelectric cell aimed at the eyelid to elicit the eye blink reflex. For EMG measurement, two electrodes are placed under the right eye. The first is aligned with the pupil, while the other is positioned immediately laterally. EMG activity is assessed using the SR-Labs equipment. This depends on whether the subject is comfortable with electrodes placed on their skin. Heart rate and galvanic skin resistance is also measured throughout the session using a heart rate monitor, finger electrodes and a galvanic skin response amplifier.

### Texture Perception and Discrimination (Laser-Cut Objects)

The textured novel object recognition (NORT) assay is utilized to assess texture discrimination abilities in subjects. Subjects are presented with two acrylic cubes of identical texture. Subjects are allowed to freely explore the objects in the learning phase for 30 seconds. After the explore phase, both objects are removed momentarily. Then the subject is presented with two objects again, with one of the objects being the same texture he/she had previously investigated, and a second novel textured object. While the objects have different textures, the objects will appear visually identical. After 15 seconds of object exploration, the subject is then asked to explore the objects, and state which object is new to them. For subjects who are non-verbal, pointing to the novel object would be a sufficient correct response. This test is performed for 10 consecutive pairs of objects that differ in roughness, ranging from very different textures to more similar textures. These trials are pseudorandomized.

As a control, subjects perform this same assay except the objects are wooden blocks that differ in color and shape (but have similar textures).

A second, more simplified version of this assay is the texture discrimination assay. During each trial, a subject is presented with two acrylic cubes. The subject is asked, between the two objects in front of them, which object is rougher. The subject is given 30 seconds to explore both objects and specify which object is perceptually rougher, either through verbal communication or pointing to the object. Ten trials, with object pairs varying in degrees and differences in roughness is used.

### Von Frey Fibers (Indentation Stimuli)

Mechanical detection threshold (MDT) is assessed using a standardized set of modified von Frey hairs (North Coast Medical, Gilroy, Calif.) exerting forces between 0.25 and 512 mN, as previously described. The contact area of the von Frey hairs with the skin is a rounded tip (0.5 mm in diameter) to avoid sharp edges that would facilitate nociceptor activation.

Stimulus trials are administered using the method of limits, with four alternating blocks of two descending and two ascending trials administered after a practice descending block, as previously described (Cascio et al., 2008). Each trial consists of a filament being manually lowered normal to the surface of the skin until contact was made with the skin, and continued force applied smoothly until the filament buckled once. The contact area is delineated with a plastic ring with an adhesive backing, that delineates an area of skin of about 1 cm in diameter. Testing will occur on both the hairy skin of the forearm, as well as the glabrous skin on the palm of the hand. Before each presentation, a verbal prompt is given: “pay attention now,” and after each presentation the subject will report whether or not he/she detected the applied filament.

The testing blocks begin with a filament that delivers a force of 8 g (78.431 mN), and after a positive response, the next thinner filament in the set is administered in the same manner, until two consecutive negative responses (no perception of the stimulus) are obtained. The ascending blocks begin with a filament which delivers a force of 0.005 g (0.05 mN), and after each negative response, the next (thicker) filament is administered, until two consecutive positive responses (perception of stimulus) are obtained. Control trials for which no filament is administered is are included in each block to limit false positive responses. Subjects is notified of false positive responses before continuing.

A subject's data is discarded if more than one in four catch trials resulted in false alarms, according to previously described procedures. Thresholds for perception of mechanical stimulation is calculated by averaging the values for the trial before and after the first of the two negative (or positive) responses that ended the block, across the four blocks.

### Somatosensory Temporal Discrimination Threshold (STDT)

The STDT is collected prior to PAS in Module 2 during the first study visit only. Participants is are seated in a comfortable chair, where the first portion of the test is to determine the intensity of the electrical stimuli to be used for each person for the STDT test. In order to do this, a Digitimer (Welwyn Garden City, UK) will deliver an electrical stimulus over the right index finger at very low intensities, increasing until the participant can perceive the stimulus 53,54. The intensity used to determine STDT is the minimal intensity perceived by each participant (reported verbally) in 10 out of 10 consecutive stimuli; therefore, the electrical stimulus used is as mild as possible for it to be felt 21. For the determination of STDT, pairs of electrical stimuli are delivered to the right index finger at the aforementioned intensity. The interval between the stimuli is gradually increased until participants can perceive both stimuli separately, as opposed to the sensation of only one stimulus. Participants are asked to report verbally whether they perceived a single stimulus or 2 temporally separated stimuli. The shortest interval between the two electrical stimuli at which participants can perceive both stimuli in 3 consecutive trials is considered the STDT 21,22. Participants is are asked throughout the session whether the intensity of the electrical stimuli are uncomfortable, and are free to stop the procedure at any point throughout the session, and that this will not have negative consequences (such as exclusion from other sessions or future studies).

ASD participants are recruited from clinics and the community, including outreach to the Boston Autism Consortium and the New England Asperger's Association. Local neurologists and in-network physicians will also be contacted with a letter that is mailed or e-mailed informing them of the study including a copy of the flyer that they can distribute to interested patients and their families. Patients will also be recruited using ICD-9 codes for Autism Spectrum Disorder (299.0) and Asperger's Syndrome (299.8), Clinical Query 2 using the CQ2 Data Download Tool is utilized to identify potential participants. Medical records are reviewed of the identified patients to assess for study eligibility. Provider schedules will also be accessed to identify potentially eligible participants. Once a potential subject is identified that fits the inclusion/exclusion criteria, the physician on record at BIDMC is contacted to ask for their permission to contact the subject by phone, email, or with a letter containing details of the study on behalf of the physician on record. Established research participant registries will also be utilized, including the Research Participant Registry (RPR) at Boston Children's Hospital, and the Interactive Autism Network (IAN) at the Kennedy Krieger Institute. The Berenson-Allen Center Data Repository (IRB Protocol #2010P-000169) will also be utilized to recruit ASD participants and healthy control subjects.

Normal control subjects is recruited from the community via flyers and postings on local university job boards. Individuals who respond to flyers, recruitment emails, or phone messages is contacted by telephone or sent an email link to a REDCap screening questionnaire for pre-screening.

ASD participants is recruited from clinics and the community with diagnoses of ASD in accordance with DSM-IV or DSM 5 criteria. Diagnosis is confirmed using ADOS criteria.

### Inclusion Criteria for Adult Subjects

All groups:
a. Age 18-65
b. Ability to obtain informed consent with the participant or legally authorized representative
For ASD group:
a. Clinical diagnosis of a disorder on the autism spectrum according to:
b. DSM-IV or DSM 5 criteria
For the Control group:
a. No history of ASD or other developmental delay
b. No history of ASD or other developmental delay in first-degree relatives.

### Exclusion Criteria for Adult Subjects

1. Intracranial pathology or lesion from a known genetic disorder (e.g., neurofibromatosis type I) or from acquired neurologic disease (e.g. stroke, tumor), cerebral palsy, history of severe head injury, or significant dysmorphology [the intracranial pathology associated with Tuberous Sclerosis complex (TSC) is not an exclusion criterion for TSC participants];

2. History of fainting spells of unknown or undetermined etiology that might constitute seizure

3. History of seizure or epilepsy will not be an exclusion criterion (see section B5. Possible Risks for details on risk of seizure in TMS). For neurotypical control subjects (e.g. no diagnosis of epilepsy or neurologic diagnoses) a seizure of benign etiology (e.g. febrile seizures) would not be an exclusion

4. Any progressive (e.g., neurodegenerative) neurological disorder or signs of increased intracranial pressure

5. Chronic (particularly) uncontrolled medical conditions that may cause a medical emergency in case of a provoked seizure (cardiac malformation, cardiac dysrhythmia, asthma, etc.);

6. Metal implants (excluding dental fillings) or devices such as pacemaker, medication pump, nerve stimulator, TENS unit, ventriculo-peritoneal shunt unless cleared by the responsible MD; (only for Module 2)

7. Substance abuse or dependence within the past six months;

8. Chronic treatment with prescription medications that decrease cortical seizure threshold.

9. Peripheral (diabetic) neuropathy; and

10. Pregnancy.

In addition, normal control participants is evaluated for a diagnosis of a psychiatric condition. If in the judgment of the investigator, the condition is well-controlled with stable medications, they may be included in the study. Examples include well-controlled depression or anxiety. Normal control participants is excluded from taking part in the study if they have a family history of ASD.

### EQUIVALENTS AND SCOPE

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A tactile prepulse inhibition (TPPI) assay, comprising the steps of:
(a) administering a tactile prepulse to a human subject;
(b) administering a startle stimulus to the subject; and
(c) measuring the subject's response to the startle stimulus.

2. The assay of claim 1, wherein the interval (ISI) between steps (a) and (b) of claim 1 is between about 15 milliseconds (ms) to about 1000 ms.

3. The assay of claim 1 or 2, wherein the ISI is about 250 ms.

4. The assay of any one of claims 1-3, wherein the tactile prepulse is an air puff.

5. The assay of claim 4, wherein the air puff is directed at the subject's forearm, the hairy skin on the back of the subject's hand, or the glabrous skin on the palm of the subject's hand.

6. The assay of claim 4, wherein the air puff is directed at the subject's thigh, leg, foot, or neck.

7. The assay of any one of claims 4-6, wherein the air puff has a pressure that is greater than about 0.1 pounds per square inch (PSI) and less than or equal to 10 PSI.

8. The assay of any one of claims 4-7, wherein the air puff has a pressure of about 0.9 PSI.

9. The assay of any one of claims 4-7, wherein the air puff has a pressure of about 2 PSI.

10. The assay of any one of claims 4-7, wherein the air puff has a pressure of about 3 PSI.

11. The assay of any one of claims 1-10, wherein the startle stimulus has an intensity of about 100-150 decibels (dB).

12. The assay of any one of claims 1-11, wherein the startle stimulus has an intensity of about 115 dB.

13. The assay of any one of claims 1-12, wherein the subject's response to the startle stimulus is measured by electromyography (EMG).

14. The assay of any one of claims 1-12, wherein the subject's response to the startle stimulus is measured by eye-blink reflex.

15. The assay of any one of claims 1-12, wherein the subject's response to the startle stimulus is measured by the subject's autonomic response.

16. The assay of any one of claims 1-12, wherein the subject's response to the startle stimulus is measured by pupillometry.

17. The assay of any one of claims 1-12, wherein the subject's response to the startle stimulus is measured by galvanic skin resistance.

18. The assay of any one of the preceding claims, wherein the subject is wearing a noise-canceling auditory device prior to and during the assay.

19. A method for evaluating tactile hypersensitivity and/or sensorimotor impairment in a human subject, comprising:
(a) administering to the subject a TPPI assay according to any one of the preceding claims;
(b) comparing the assay results to neuro-typical controls; and
(c) determining the degree of tactile hypersensitivity and/or sensorimotor impairment in the subject.

20. The method of claim 19, further comprising the step of:
(d) adjusting a treatment of tactile hypersensitivity and/or sensorimotor impairment.

21. The method of claim 20, wherein said adjusting comprises increasing or decreasing the dosage or duration of the treatment.

22. The method of claim 20, wherein said adjusting comprises changing the type of treatment.

23. The method of claim 20, wherein the subject has been diagnosed with Autism Spectrum Disorder (ASD), Rett Syndrome (RTT), Phelan McDermid syndrome (PMS), Fragile X Syndrome, Neurofibromatosis, or Tuberous Sclerosis complex.

24. The method of claim 20, wherein the treatment is a pharmacological treatment.

25. The method of claim 22, wherein the pharmacological treatment comprises administering to the subject a GABA_{A} agent.

26. The method of claim 22, wherein the GABA_{A} agent is peripherally restricted.

27. The method of any one of claims 19-26, further comprising administering to the subject one or more screening/assessment procedures selected from:
(1) demographics and/or family history questionnaire;
(2) neurological and physical exam including screening for peripheral neuropathy;
(3) medical history questionnaire;
(4) medication review;
(5) inclusion/exclusion criteria review;
(6) pregnancy test and menstrual history questionnaire for females of childbearing potential;
(7) Transcranial Magnetic Stimulation (TMS) safety screening;
(8) Mini Mental State Evaluation (MMSE);
(9) verbal and/or non-verbal intelligence testing;
(10) Brain-derived neurotrophic factor (BDNF), Catechol-O-methyltransferase (COMT), and/or apolipoprotein E (APOE) genotyping;
(11) Autism Diagnostic Observation Schedule (ADOS);
(12) Autism Spectrum Quotient (AQ);
(13) Pupil dilation; and
(14) Eyes Test.

28. The method of any one of claims 19-27, further comprising administering to the subject an acoustic prepulse inhibition assay (APPI).

29. The method of any one of claims 19-28, wherein the neuro-typical controls are age-matched, gender-matched, and/or intelligence quotient-matched (IQ-matched).

30. The method of any one of claims 19-29, wherein the neuro-typical controls have no history of ASD or other developmental delay in themselves or in any known first-degree relatives.

31. The method of any one of claims 19-30, wherein the subject is 18-65 years of age.

32. The method of any one of claims 19-30, wherein the subject is a child.

33. The method of any one of claims 19-32, wherein the subject has been clinically diagnosed with a disorder on the ASD spectrum according to the Diagnostic and Statistical Manual of Mental Disorders (DSM)-IV or DSM-5.

34. The method of any one of claims 19-33, wherein an assay result indicating increased tactile hypersensitivity and/or sensorimotor impairment relative to neuro-typical controls indicates the presence of a disorder in the subject, wherein the disease or disorder is selected from ASD, RTT, PMS, Fragile X syndrome, Neurofibromatosis, and Tuberous Sclerosis complex.

35. The method of claim 34, wherein the degree of tactile hypersensitivity and/or sensorimotor impairment relative to the neuro-typical controls indicates the severity of the disorder in the subject.

36. The method of claim 34 or 35, wherein the ASD is syndromic or non-syndromic.

37. A method for assessing the efficacy of a treatment for a disorder characterized by tactile hypersensitivity and/or sensorimotor impairment comprising:
(a) evaluating a subject using the method of any one of claims 19-36;
(b) administering a treatment to the subject;
(c) re-evaluating the subject using the method used in step (a); and
(d) assessing the efficacy of the treatment of step (b) by comparing the results of steps (a) and (c).

38. The method of claim 37, further comprising the step of:
(e) adjusting the treatment.

39. The method of claim 38, wherein said adjusting comprises increasing or decreasing the dosage or duration of the treatment.

40. The method of claim 38, wherein said adjusting comprises changing the type of treatment.

41. The method of claim 37, wherein the treatment is a pharmacological treatment.

42. The method of claim 41, wherein the pharmacological treatment comprises administering to the subject a GABA_{A} agent.

43. The method of claim 36, wherein the GABA_{A} agent is peripherally restricted.

44. The method of any one of claims 37-43, wherein the disorder is Autism Spectrum Disorder (ASD), Rett Syndrome (RTT), Phelan McDermid syndrome (PMS), Fragile X Syndrome, Neurofibromatosis, or Tuberous Sclerosis complex.

45. A system or apparatus for performing an assay of any one of claims 1-18, or a method of any one of claims 19-44, the system or apparatus comprising:
(a) means for administering a tactile prepulse to a human subject;
(b) means for administering a startle stimulus to the subject; and
(c) means for measuring the subject's response to the startle stimulus.
